(19) United States
(12) Patent Application Publication
(10) Pub. No.: US 2018/0292346 A1
(43) Pub. Date: Oct. 11, 2018

(54) SYSTEM FOR LIBERATING AND MEASURING NITRIC OXIDE

(71) Applicant: LifeHealth, LLC, Roseville, MN (US)

(72) Inventors: Michael E. Collison, Eagan, MN (US); Amanda Marie Franey, Princeton, MN (US); Gregory Scott Buell, Plymouth, MN (US); Lyna Anderson, Eagan, MN (US); Jonathan S. Stamler, Shaker Heights, OH (US)

(21) Appl. No.: 15/949,955

(22) Filed: Apr. 10, 2018

Related U.S. Application Data
(60) Provisional application No. 62/483,720, filed on Apr. 10, 2017.

(51) Int. Cl. G01N 27/327; G01N 27/30; G01N 27/40; G01N 27/42; G01N 33/49; B01L 3/00

(52) U.S. Cl. CPC G01N 27/3277; G01N 27/301; G01N 27/40; G01N 27/42; G01N 33/4925; B01L 2200/026; B01L 3/502753; B01L 2300/0645; B01L 2300/0681; B01L 2300/18; B01L 3/502715

(57) ABSTRACT

A gaseous nitric oxide (NO) sensor having a working electrode configured such to induce NO molecules proximate the working electrode to undergo an electrochemical oxidation reaction. The electrochemical oxidation reaction transfers electrons from the NO molecules to the working electrode, creating a measurable current corresponding to the NO concentration within the fluid sample. An external membrane can be deposited onto the working electrode to separate the fluid sample from the working electrode. The external membrane can comprise an ionomer selectively permitting diffusion of NO molecules through the external membrane from the fluid sample to the working electrode. In an example, the fluid sample can be heated to a predetermined temperature to facilitate dissociation of NO from its hemoglobin bonded forms proximate the external membrane.

### PRIORITY

This application claims the benefit of priority of U.S. Provisional Application Ser. No. 62/483,720, filed on Apr. 10, 2017, and which is incorporated by reference herein in its entirety.

### TECHNICAL FIELD

This document pertains generally, but not by way of limitation, to systems and related methods for in-situ liberation and measurement of chemically-bonded, complexed, or coordinated nitric oxide.

### BACKGROUND

Disruptions in blood flow and oxygen delivery can be indicators of many common ailments affecting the heart, lung, and vasculature. In particular, these dysfunctions are exhibited at the microvascular level where oxygen exchange between blood and respiring tissues occurs. Oxygen exchange at the microvasculature level is a finely regulated process involving hemoglobin (“Hb”), red blood cells (“RBCs”), and the gases of the respiratory system: nitric oxide (“NO”), oxygen and carbon dioxide. Due to the three-gas respiratory cycle (NO, oxygen and carbon dioxide), blood flow is the primary determinant of oxygen delivery rather than blood oxygen content.

Blood flow at the microvasculature level is primarily regulated by physiological oxygen gradients where changes in oxygen saturation of Hb depend on regulated vasoconstriction or vasodilation to match oxygen delivery with local metabolic demand. During venous circulation of RBCs, NO binds to the heme iron of deoxy T-state (tense) Hb in RBCs to generate HbFeNO (iron nitrosylhemoglobin), which, during oxygenation in the lungs, generates oxy R-state (relaxed) Hb and SNOHb (S-Nitrosohemoglobin). At the arterial periphery respiring tissues cause the blood oxygen content to transition from high to low partial pressure. This change in blood oxygen concentration causes oxy R-state Hb to deoxygenate and transition to the deoxy T-state Hb structure. This oxygen-sensitive structural change of Hb causes release of NO from SNOHb and export of S-Nitrosothiols (“SNOs”) from RBCs to induce SNO-based vasodilatory activity and increase blood flow. By exporting SNOs as a function of Hb deoxygenation, RBCs dispense NO vasodilatory activity in direct proportion to regional oxygen requirements. As SNO-Hb causes oxygen-dependent increases in blood flow in system arterioles, RBCs facilitate the uptake and delivery of oxygen to respiring tissues.

As the respiratory cycle is dependent on NO, NO dysfunction at the microvasculature level can be indicative of many ailments. Similarly, as NO dysfunction can affect the levels and impair bioavailability of SNOHb, irregular levels and bioavailability of SNO can also be indicative of many ailments. However, while NO dysfunction at the microvasculature level is a significant indicator of common ailments, simple diagnostic tests for NO dysfunctions in oxygen homeostasis are lacking.

### OVERVIEW

The present inventors have recognized, among other things, that a problem to be solved can include providing rapid and efficient point-of-care (“POC”) measurement of chemically bound NO levels, in covalently bonded SNOHb and heme iron-coordinated HbFeNO forms, within RBCs of a patient sample. In an example, the present subject matter can provide a solution to this problem, such as by a gaseous NO sensor having a working electrode configured such that NO molecules proximate the working electrode undergo an electrochemical oxidation reaction. The electrochemical oxidation reaction transfers electrons from the NO molecules to the working electrode to create a measurable current corresponding to the NO concentration within the fluid sample. An external membrane can be deposited over the working electrode to separate the fluid sample from the working electrode. The external membrane can comprise an ionomer selectively permitting diffusion of NO molecules through the external membrane from the fluid sample to the working electrode. In an example, the fluid sample can be heated to a predetermined temperature to facilitate dissociation of NO from its sulfur-bound form (SNOHb) and its heme-complexed form (HbFeNO) proximate the external membrane.

The external membrane ionomer can comprise a perfluorinated polymer having highly dissociated, negatively charged sulfonic acid functional groups. The dissociated sulfonic acid functional groups (sulfonates) can form highly polar, fully hydrated, hydrophilic clusters interspersed with hydrophobic semi-crystalline regions of perfluorinated polymer backbones. The hydrophobic character of semi-crystalline regions or the polarity and charge density of the sulfonated hydrophilic clusters can modify the lipid bilayer membrane of RBCs and favorably dispose the membranes for release of intracellular, cytosolic Hb to the extracellular space immediately adjacent to the external membrane of the NO sensor. The polar, hydrophilic clusters can capture and immobilize a multitude of cytosol-free, extracellular Hb protein molecules that have been released by rupture and hemolysis of fluid sample RBCs.

This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a schematic top view of a sensor array according to an example of the present disclosure.

FIG. 2 is a schematic side view of a sensor array according to an example of the present disclosure.

FIG. 3 is a side view of a test cartridge according to an example of the present disclosure.

FIG. 4 is a schematic top view of a test cartridge according to an example of the present disclosure.

FIG. 5 is a perspective view of a reader system for use with the test cartridge according to an example of the present disclosure.

FIG. 6 is a schematic diagram illustrating a method of calibrating a sensor array and testing a biological sample according to an example of the present disclosure.

FIG. 7 is a schematic diagram illustrating preparation of a biological sample for testing by a sensor array according to an example of the present disclosure.

### DETAILED DESCRIPTION

Blood flow at the microvasculature level is primarily regulated by physiological oxygen gradients where changes in oxygen saturation of Hb depend on regulated vasoconstriction or vasodilation to match oxygen delivery with local metabolic demand (hypoxic vasodilation). During venous circulation of RBCs (hypoxic low oxygen tension), NO is complexed by the heme iron of deoxygenated T-state (tense state) Hb in RBCs to generate hemoglobin iron-nitrosyl (heme iron-coordinated HbFeNO). While transiting the lungs (high oxygen tension) RBC Hb is oxygenated (oxy-Hb), which causes the protein structure of Hb to convert from T-state to R-state (relaxed state). This oxy-Hb structural change causes the conversion of iron-coordinated HbFeNO to covalently bound S-nitrosylated Hb (SNO-Hb). In the resulting SNOHb species, NO is covalently bound to the thiol sulfur of the Cys93 residue of a Hb β subunit (βCys93). The auto-S-nitrosylated βCys93 residue remains hydrophobically buried in the R-state oxy-Hb configuration and, thus, is unavailable for vasodilatory activity. During deoxygenation of the Hb (deoxy-Hb) at the arterial periphery respiring tissues reduce partial pressures of oxygen. Under these low oxygen tensions, Hb releases oxygen causing the Hb quaternary structure to convert from R-state oxy-Hb to T-state deoxy-Hb. This Hb structural change, in turn, increasingly exposes the NO group on βCys93 to aqueous solvent thereby promoting covalent S—N bond cleavage and concomitant release of gaseous NO. Within the interior microenvironment of the Hb protein, gaseous NO released from the βCys93 residue can transfer directly to the aqueous cytosol or it can intramolecularly auto-coordinate at the heme iron forming transitory iron-complexed HbFeNO. With continuing circulation to lower oxygen tension tissue beds transitory iron-complexed HbFeNO can also release gaseous NO into the aqueous cytosol. Once released to the aqueous RBC cytosol reactive gaseous NO is subject to at least three predominate reaction paths. In the first, NO gas can quickly react with cytosolic Hb proteins as described above. In the second, NO gas can be oxidized to various species, most principally nitrite and nitrate. In the third path, NO gas is covalently bound by cytosolic thiolic amino acids (principally glutathione) to form low molecular mass S-Nitrosoglutathione (GSNO). By the first and second reaction paths, cytosolic gaseous NO vasodilatory activity is extinguished. In the third reaction path (covalent formation of LMM-GSNO), cytosolic NO can be preserved in a stabilized bioactive form that can be transported to the cytosolic side of the RBC membrane. At the RBC membrane cytosolic LMM-GSNO transfers NO bioactivity to thiols of membrane-bound Hb which, in turn, transfers NO bioactivity to thiols of membrane-bound Anion-Exchange protein 1 (AE-1). The AE-1 protein, in turn, exports NO bioactivity to the extracellular space, primarily through transfer to plasma cysteine thiols, thereby forming both the LMM-SNO S-Nitrosocysteine (SNOCys) and the high molecular mass SNO (HMM-SNO) species S-Nitrosoalbumin (SNOA1b). NO bioactivity can be preserved in the plasma compartment via the relatively stable SNOCys and SNOAlb species during transport to the endothelial lining of blood vessels where NO manifests its vasodilatory activity.

RBCs export preserved NO bioactivity, in the forms of LMM-SNOs and HMM-SNOs, to deliver NO- and SNO-based vasodilatory activity to the peripheral circulation in an oxygen-dependent manner. This NO/SNO-based vasodilatory activity increases flow of oxygenated blood and delivery of oxygen to hypoxic respiring tissues. By exporting SNOs as a function of localized decreases in oxygen tension and Hb deoxygenation, RBCs dispense NO/SNO vasodilatory activity in direct proportion to regional oxygen demand (hypoxic vasodilation). As SNO-Hb causes oxygen-dependent increases in blood flow in systemic arterioles, RBCs and erythrocytic Hb-associated NO (SNOHb, HbFeNO) facilitate the delivery of oxygen to hypoxic regions of respiring tissues.

Because gaseous NO is a reactive free radical, the complex NO reaction paths and the formation of stable, covalently bonded LMM- and HMM- SNO and iron-coordinated HbFeNO species are required to effect oxygen-dependent release of stable NO bioactivity from oxygen-sensitive Hb proteins and transport of the same from RBC-encapsulated Hb, through the RBC cytosol and membrane, and through the blood plasma to the endothelial linings of blood vessels. At the endothelial cells, the vasodilatory signaling of NO can precipitate blood vessel dilatation, increasing blood flow, and increasing delivery of oxygen and nutrients to hypoxic, respiring tissues.

In the biological milieu, the stable covalently bound forms of NO (LMM- and HMM-SNOs) and stable metal-coordinated forms of NO (e.g. HbFeNO) are not amenable to practicable direct measurement near or at the point-of-use (POU) or POC. By contrast, gaseous NO released from its stable bound forms (SNOs, HbFeNO, etc.) can be more easily measured by electrochemical NO gas sensors and POU/POC electrochemical diagnostic analyzer systems.

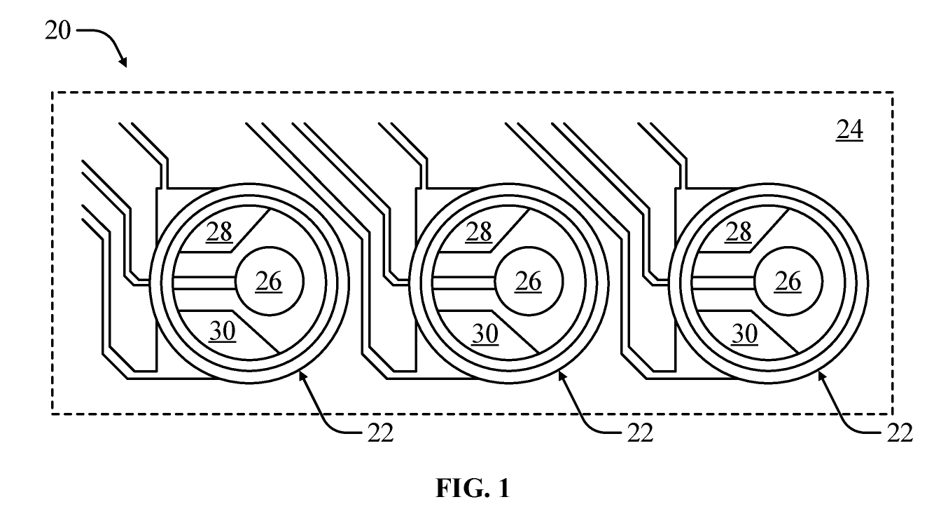

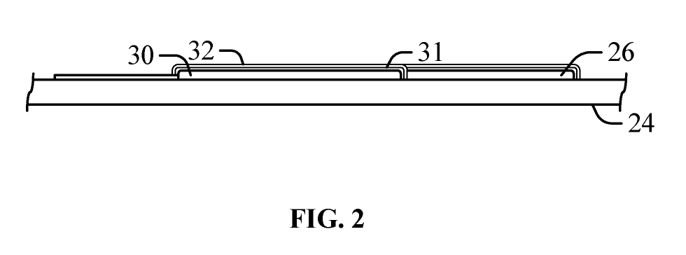

As depicted in FIGS. 1 and 2, a nitric oxide (“NO”) sensor array 20, according to an example of the present disclosure, can include at least one NO sensor 22 positioned on a substrate 24. In an example, each NO sensor 22 can include a working electrode 26, an auxiliary electrode 28, and a reference electrode 30. In an example, the working electrode 26 and the auxiliary electrode 28 can comprise platinum while the reference electrode 30 can comprise silver. An external membrane 32 can be deposited over at least the working electrode 26 to separate at least the working electrode 26 from the sample. The external membrane 32 can separate at least the working electrode 26 to control diffusion of NO molecules liberated from the sample for tuning the NO measurement range and detection limits of the NO sensor 22.

NO molecules that diffuse through the external membrane 32 undergo an electrochemical oxidation reaction at the working electrode 26 that transfers electrons from the NO molecules to the working electrode 26 generating a measurable current. In an example, the electrochemical oxidation reaction can comprise:

NO→NO⁺+e⁻ (1)

NO⁺+OH⁻→HNO₂ (2)

HNO₂+H₂O→NO₃⁻+2e⁻+3H⁺ (3)

The electrode array 22 can comprise an amperometric measurement circuit having a potential between the reference electrode 30 and the working electrode 26 to create a working electrode bias at the working electrode 26. In an example, the working electrode bias can be about 0.6 to 0.9 V versus a silver/silver chloride reference potential. The working electrode bias induces the NO to undergo the electrochemical oxidation reaction after passing through the external membrane 32 and upon reaching the working electrode 26.

In an example, the current from the electrochemical oxidation reaction can flow to the auxiliary electrode 28 thereby maintaining the potential between the working electrode 26 and the reference electrode 30 inducing the electrochemical oxidation reaction at the working electrode 26. An analyzer circuit can monitor and control the distribution of current created the electrochemical oxidation reaction to maintain the working electrode bias at a constant level irrespective of the current generated by the electrochemical reaction.

### Internal Layer

The surface of the reference electrode can be maintained at a constant phase boundary potential to maintain the surface interfacial potential of the working electrode at a constant potential. In an example, the interfacial potential can be maintained at the constant potential by depositing an internal layer 31 over at least the reference electrode 30. The internal layer 31 can comprise a hydrophilic polymer matrix comprising, but not limited to polyvinyl alcohol (“PVA”); polyvinylpyrrolidone (“PVP”); polyvinyl-acetate (“PVAc”); polyhydroxy-ethyl-methacrylate (“pHEMA”); or combinations thereof. In an example, the external membrane 32 can be deposited over the internal layer 31.

For silver reference electrodes 30, a silver-silver chloride layer can be formed on a surface of the reference electrode 30. The internal layer 31 can also comprise chloride salts including, but not limited to potassium chloride, sodium chloride, calcium chloride, or magnesium chloride. The internal layer 31 can also comprise an acid including, but not limited to an acid hydrochloric acid, nitric acid, formic acid, or acetic acid. The chloride salt and acid provide a controlled concentration of chloride ions at the surface of the reference electrode 30 maintaining interfacial phase boundary potential of the reference electrode 30. The controlled interfacial phase boundary potential of the reference electrode 30 maintains the interfacial phase boundary potential of the working electrode 26 at the potential for inducing the NO reaching the working electrode to undergo the electrochemical oxidation reaction.

### External Membrane

The external membrane 32 can promote diffusion of NO molecules from the sample to the working electrode 26. The external membrane 32 can comprise ion-containing polymers (“ionomers”) including, but not limited to sulfonated linear polyester ionomers; long-side-chain (“LSC”) sulfonated tetrafluoroethylene fluoro ionomers; perfluorinated ionomer; short-side-chain (“SSC”) perfluoro alkoxy ionomers, amphiphilic polymers, zwitterionic polymers, siloxane polymers, urethane polymers, polymers with chemically tunable physical and chemical properties (e.g. surface energy, hydrophobicity/hydrophilicity, electrostatic charge, acidity/basicity), and other ionomers.

In an example, the external membrane 32 can comprise perfluorinated ionomer having sulfonic acid functional groups. The acidic sulfonic acid functional groups will become ionized and negatively charged at physiologic pHs. The ionized functional groups will associate to form polar, hydrophilic, hydrated clusters that are interspersed with highly hydrophobic semi-crystalline regions of perfluorinated polymer backbones. In an example, the polar, hydrophilic clusters can have a diameter of about 30 to about 50 nm. The hydrophobic character of semi-crystalline perfluorinated polymer backbone regions and/or the polarity and charge density of the sulfonated hydrophilic clusters can capture and immobilize sample RBCs and modify their lipid bilayer membranes favorably disposing the membranes to rupture and release of intracellular, cytosolic Hb to the extracellular spaces immediately adjacent to the NO sensor external membrane. The negatively charged sulfonate moieties and associated hydrophilic, hydrated clusters of hydrated perfluorinated ionomers attract, and capture/immobilize/retain RBCs and/or cell-free Hb protein molecules at the surface of the external membrane 32. Both hemoglobin and intact red blood cell membranes can have net negative charges at a physiological pH of 7.4.

In an example, the solution containing the biological sample and measurement temperature can be changed to alter the net charges of the molecules on the RBC membranes and Hb proteins such that the interactions with the external membrane effect different NO cleavage and release profiles from the Hb-bound forms. The solution changes can include, but are not limited to pH, ionic strength, buffer capacity, identity, and concentration of ionic and/or other dissolved species. These solution changes can facilitate rupture and release of intracellular, cytosolic Hb and/or modification of at least one of the quaternary and/or tertiary structural conformations of extracellular Hb protein and release measurable NO gas from its Hb-bound form. Liberated NO molecules can undergo side reactions (e.g. oxidation by dissolved oxygen to nitrite or nitrate; auto-capture of NO by hemoglobin; capture and binding by thiols) that compete with the electrochemical oxidation reactions necessary for measurement by the working electrode 26. By capturing and immobilization of these species at the surface of the external membrane 32, the potential for side reactions and the diffusion distance and time for liberated NO molecules to travel to the surface of the external membrane 32 is reduced. The reduced inhibitions on freed NO molecules diffusing into the external membrane 32 lowers NO detection limits, increases NO sensitivity, and increases NO measurement accuracy and precision.

The highly hydrated, ionized sulfonate ionic clusters are integrally interspersed among the semi-crystalline highly hydrophobic perfluorinated polymer backbone regions. The arrangement of the ionic clusters and the semi-crystalline regions causes large and abrupt changes in hydrophilic and hydrophobic characteristics of the perfluorinated polymer. The amphiphilic, molecular scale spatially abrupt variations in polar hydrophilic and hydrophobic characteristics disrupt the normal biological structures of red blood cell membranes and cell-free Hb proteins. Both Hb and RBCs have hydrophobic regions capable of strong interactions with hydrophobic materials and surfaces.

The normal biological structures of intact RBCs membranes and native Hb proteins depend strongly on self-ordering of biochemical components through non-covalent ionic, hydrophilic, and hydrophobic interactions. The integrity of lipid bilayer membranes of RBCs can be disrupted by exposing the RBCs to hydrophobic surfaces. The rupture of the RBCs directly exposes cytosolic Hb to direct interactions with the external membrane 32. The disruptions make Hb-bound NO moieties (e,g. SNO, FeNO) more accessible to hydrophobic perfluorinated polymer backbone regions and aqueous sample interactions. The disruptions also modify the Hb NO bond and complexation strengths and energies to facilitate cleavage and release of NO gas from its Hb-bound forms proximate near the external membrane 32. Specifically, the disruptions modify the S—N bond strength and Fe—No complexation strength of the SNOHb and HbFeNo species, respectively. The amphiphilic character of the NO sensor's external membrane models the amphiphilic character of detergents that readily denature most proteins, including the Hb protein. Accordingly, the amphiphilic nature of the NO sensor's external membrane can be designed to modify at least one of the Hb protein's quaternary and/or tertiary structural conformations and favorably alter the S—NO bond and Fe—NO complexation energies of Hb-bound NO in order to facilitate cleavage and release of measurable NO gas from the same. The altered bond and complexation strengths permit selective and efficient cleaving and release of NO from SNOHb and HbFeNo species proximate the external membrane 32. Releasing the NO proximate to the external membrane 32 minimizes losses of released NO in competing side reactions, which thereby enhances detection limits, sensitivities, dynamic ranges, accuracies, and precision of in situ, real-time measurements of the released NO molecules by the NO sensor 22.

The external membrane 32 can limit or restrict electrochemically oxidizable interfering molecules from reaching the working electrode 26. The interfering molecules can comprise, among others, nitrite, acetaminophen, ascorbic acid, uric acid, dopamine, or carbon monoxide. The interfering molecules can be present in the biological sample. The external membrane 32 can prevent diffusion of the interfering molecules through the external membrane 32 to reach the working electrode 26.

In an example, the thickness of the external membrane 32 can alter the diffusion of NO molecules through the external membrane 32 and control of the diffusion of interfering molecules through the external membrane 32. Increasing the thickness of the external membrane 32 enables greater selectivity of which molecules pass through the external membrane. Similarly, increasing the thickness of the external membrane 32 reduces or slows the NO molecules to reach the working electrode 26 thereby reducing the sensitivity and dynamic range of the NO sensor 22.

### Cartridge

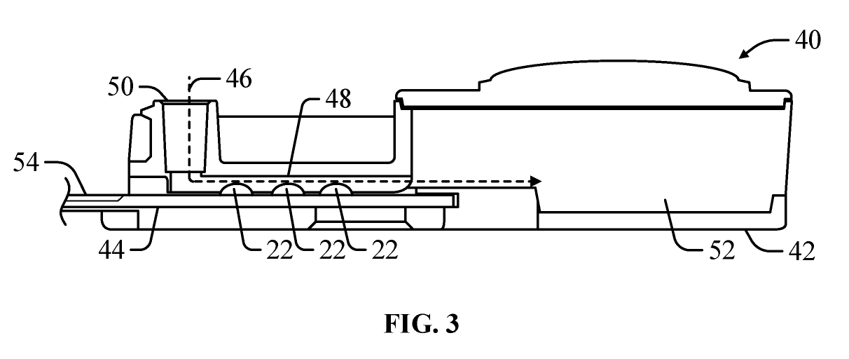

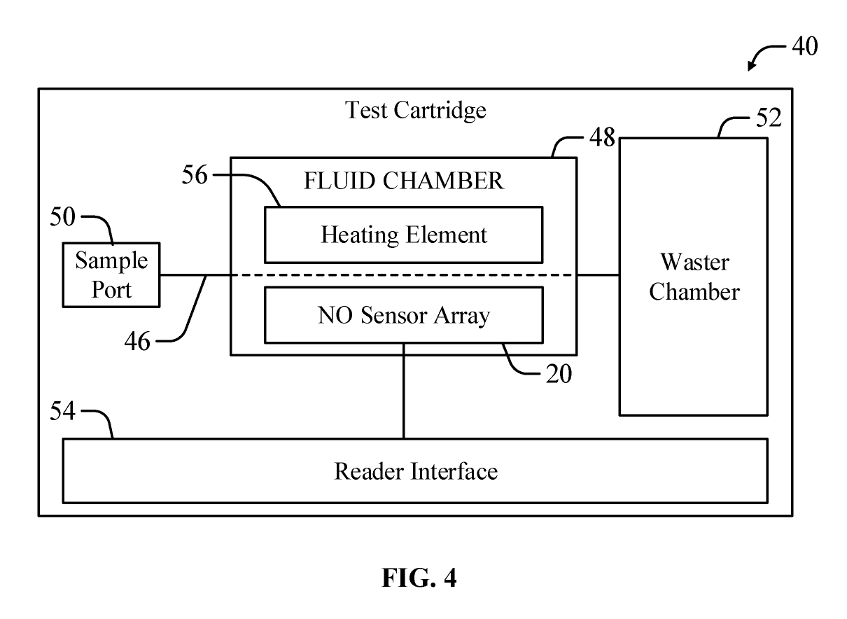

As depicted in FIGS. 3-4, a cartridge 40 for evaluating NO concentration within a biological sample, according to an example of the present disclosure, can comprise a cartridge body 42 and a sensor chip 44 having at least one NO sensor array 20. As depicted in FIG. 2, in an example, the sensor chip 44 comprise at least three NO sensors 22. The cartridge body 42 can define a flow path 46 intersecting a fluid chamber 48, wherein a portion of the sensor chip 44 is received within the cartridge body 42 such that the NO sensors 22 are positioned in the fluid chamber 48.

The flow path 46 can extend from a sample port 50 through the fluid chamber 48 and to a waste chamber 52. In operation, a biological sample, a calibration fluid, or a liquid quality control fluid can be fed into the sample port 50 and passed across the NO sensors 22 positioned within the fluid chamber 48. In an example, the fluid within the fluid chamber 48 can be processed to liberate NO molecules for diffusion through the external membrane 32 to the working electrode 26 of the NO sensors 22.

In an example, the cartridge 40 can include a heating element 56 to maintain the temperature within the fluid chamber 48 at a predetermined temperature or above a predetermined threshold, when the cartridge is inserted into a reader. In at least one example, the fluid chamber 48 can be maintained at a temperature between about 40° C. and about 60° C. The heating element 56 can be positioned on the sensor chip 44 that can be adjusted to maintain the temperature within the fluid chamber 48 at the predetermined temperature.

Rates of RBC hemolysis and release of cytosolic Hb into the extracellular spaces can be temperature dependent. Hemolysis does not occur at temperatures less than about 37° C. and can occur rapidly at temperatures exceeding 45° C. with accompanying gross changes in cellular morphology. The activation energy for hemolysis is 80 kcal/mole at temperatures exceeding 45° C. and is characteristic of protein denaturation and enzyme inactivation suggesting that these processes contribute to hemolysis at these temperatures. At temperatures between 38° C. and about 45° C., hemolysis occurs with an activation energy of about 25-30 kcal/mole or about 29 kcal/mole suggesting that hemolysis is occurring from a different mechanism than at higher temperatures. Specifically, hemolysis can be the result of the critical bilayer assembly temperature of cell membranes. The unilamellar state of the membrane is stable at 37° C. but is transformed to a multibilayer when the temperature is raised, hemolysis and release of cytosolic Hb results because formation of the multibilayer requires exposing lipid-free areas of the erythrocyte surface.

Human Hb is a globular tetrameric protein comprised of two non-covalently bonded α- and two β-subunits of similar structure and size. The subunits can comprise well-defined three-dimensional structures formed by multiple noncovalent interactions such as hydrogen bonds, salt bridges, and hydrophobic interactions. Since the four chains are stabilized by weak noncovalent interactions, the biologically active tetramers can easily undergo dissociation and denaturation. Exposure to temperatures around 50° C. can lead to rapid irreversible denaturation followed by precipitation. When in the oxy-Hb state, both iron-coordinated HbFeNO and covalently bound SNOHb groups can be oriented so that the groups are held in hydrophobic pockets protected from interaction with aqueous solvent. Upon denaturation, temperature-induced or otherwise, the Hb protein structure unfolds exposing both HbFeNO and SNOHb groups to aqueous solvent. This aqueous exposure modifies the NO coordination and covalent bond energies, respectively, of the two NO-bound groups.

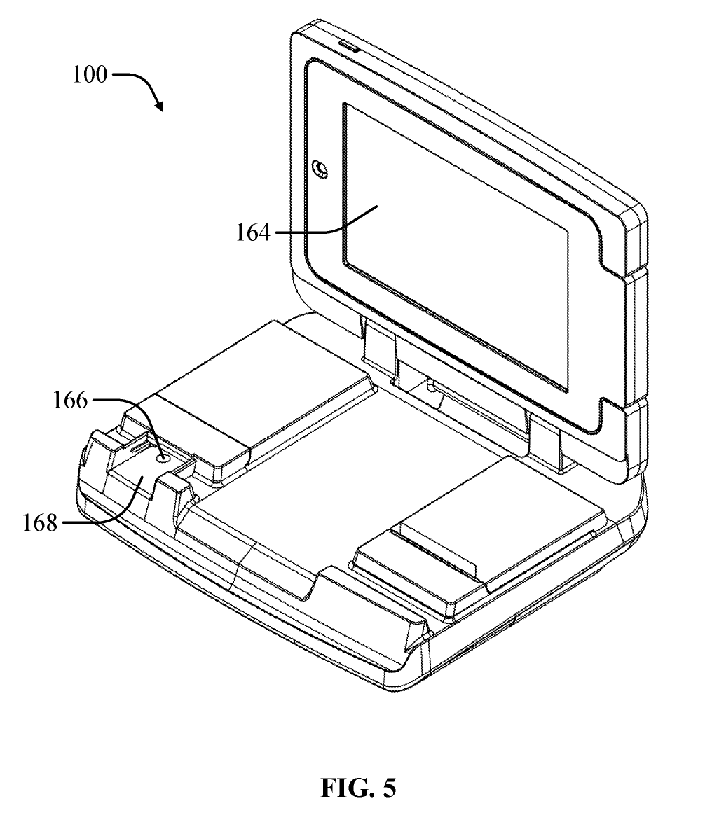

In an example, the sensor chip 44 can include at least one interface 54 for contacting corresponding interfaces of a reader system 100 for receiving or monitoring the current generated at the working electrode 26 by the electrochemical oxidation reaction. The reader system 100 is shown in FIG. 5 and can also be referred to as an instrument or electrochemical analyzer. The reader system 100 can have an analyzer circuit for monitoring the current generated at the working electrode 26 to determine the NO concentration in the sample. In an example, the reader system 100 can include, among other things, a display 164 for displaying the measurements and analysis of the measurements.

In an example, the reader system 100 can include an infrared temperature sensor 166, contained within a cartridge port 168 of the system 100, that measures the temperature within the fluid chamber 48 of the cartridge 40. The infrared temperature sensor 166 can be used to adjust the heating element 56 to maintain the fluid chamber 48 of the cartridge 40 at the predetermined temperature. In an example, the heating element 56 can be a resistive heating element wherein the current supplied to the heating element 56 is varied to alter the temperature supplied from the heating element 56.

In operation, the cartridge 40 can be inserted into the cartridge port 168 of the reader system 100 to engage the at least one interface 54 of the sensor chip 44 with the corresponding interfaces of the reader system 100. A syringe or other container containing calibration fluid or biological sample can be attached to the sample port 50 of the cartridge 40 to feed the contained fluid into the fluid chamber 48 through the flow path 46. In this configuration, the sample port 50 can comprise a luer port for coupling the syringe to the cartridge 40. In an example, the calibration fluid can be pre-stored within the fluid chamber 48 until the cartridge 40 is to be used.

In an example, the calibration fluid or biological sample can be heated to a predetermined temperature with the heating element 56. The heating of the calibration fluid or biological sample can induce liberation of the NO proximate to the working electrode 26 such that NO diffuses through the external membrane 32 to the working electrode 26.

The NO sensors 22 positioned within the fluid chamber 48 evaluate the fluid within the fluid chamber 48 to measure the concentration of NO within the fluid. Appropriate algorithms are applied by the reader system 100 to the raw data collected from the NO sensors 22. Calibration measurements from the calibration fluid can be used to modify the NO measurements made of the biological sample.

### Preparation of Sample

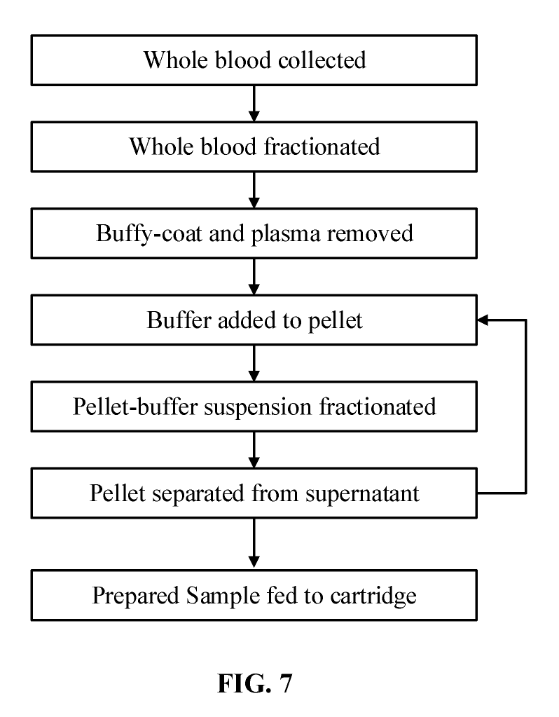

As depicted in the flow chart of FIG. 7, in an example, a whole blood sample can be prepared prior to being administered into the cartridge 40.

The whole blood sample is centrifuged and washed with a phosphate buffered saline solution containing ethylene diamine tetra acetic acid chelating agent (“PBS-EDTA”). In an example, the PBS-EDTA solution can be 24 mM phosphate buffered saline containing 0.1 mM ethylene diamine tetra acetic acid chelating agent at a pH of 7.4 at 20° C. The RBCs can be isolated from the remainder of the whole blood sample by centrifugation, for example, at 3,000 g for about 2 to about 5 minutes. After fractionation of the whole blood sample, the plasma and buffy-coat layer are removed and volumes of PBS-EDTA (e.g. 3 to 4 volumes) to the RBCs. The PBS-EDTA is intermixed with the RBCs by gentle mixing on a rotating platform for about 1 to about 3 minutes. The RBCs can be isolated in the mixed solution by centrifugation, for example, at 3,000 g for about 2 to about 5 minutes. After the centrifugation, the supernatant can be removed and discarded. The PBS-EDTA wash steps are repeated from 1 to 5 more times.

The red blood cell pellets are placed in a suspension by adding fresh PBS-EDTA, which is gently rocked to re-suspend the RBCs within the saline solution. In an example, the final hematocrit measure of the suspension sample solution is about 35% to about 55%.

### Calibration fluid

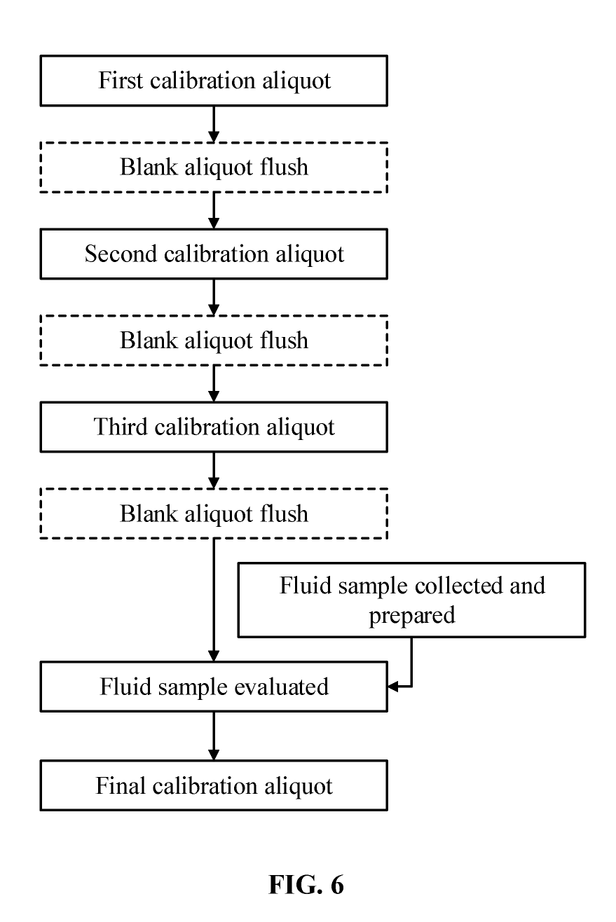

FIG. 6 illustrates a calibration method. The calibration fluid can comprise a buffered saline solution containing an NO donor formulated to provide a known NO concentration. In an example, the calibration fluid can comprise PBS-EDTA with a PROLI-NONO-ATE formulated NO donor, wherein the NO donor comprises a known NO concentration. The NO sensor 20 evaluates each calibration fluid to measure an actual NO concentration, which is compared with the expected known NO concentration for the particular calibration fluid.

In an example, multiple calibration fluids can be sequentially fed into the cartridge 40 with each calibration fluid having a different NO concentration. In this configuration, the multiple calibration fluids can comprise an aliquot of a first calibration fluid can be provided with a known NO concentration corresponding to a bottom end of the expected measurement range for the biological sample. Similarly, the multiple calibration fluids can comprise an aliquot of a second calibration fluid can be provided with a known NO concentration corresponding to a top end of the expected measurement range for the biological sample. In an example, the multiple calibration fluids can comprise an aliquot of a third calibration fluid can be provided with a known NO concentration corresponding within the expected measurement range for the biological sample. Each aliquot of calibration fluid pushes the prior aliquot of calibration fluid from the fluid chamber 48 and into the waste chamber 52. In an example, the fluid chamber 48 can be flushed with a blank saline solution (e.g. PBS-EDTA) without a NO donor between aliquots of calibration fluid containing NO donor.

In an example, at least one aliquot of calibration fluid can be administered after the biological sample has been evaluated by the NO sensors 22. The calibration fluid flushes the biological sample from the fluid chamber 48 and into the waste chamber 52. The additional calibration fluid is then evaluated to provide an additional calibration point (final calibration) for evaluation of the NO measurements obtained from the biological sample.

### VARIOUS NOTES & EXAMPLES

Example 1 is a nitric oxide (NO) sensor for measuring NO concentration within a fluid sample, the nitric oxide sensor comprising: a working electrode inducing NO molecules in the fluid sample to undergo an electrochemical oxidation reaction transferring electrons from the NO molecules to the working electrode, thereby inducing a measurable current corresponding to the NO concentration within the fluid sample; and an external membrane deposited over the working electrode separating the fluid sample from the working electrode, wherein the external membrane comprises an ionomer selectively permitting diffusion of NO molecules through the external membrane from the fluid sample to the working electrode.

In Example 2, the subject matter of Example 1 optionally includes a reference electrode having a surface interfacial potential to create a working electrode bias at the working electrode, the working electrode bias induces the NO molecules to undergo the electrochemical oxidation reaction upon diffusing through the external membrane.

In Example 3, the subject matter of Example 2 optionally includes an internal layer comprising a hydrophilic polymer matrix deposited over the reference electrode.

In Example 4, the subject matter of any one or more of Examples 2-3 optionally include an auxiliary electrode for receiving the current generated from the NO molecules undergoing the electrochemical oxidation reaction.

In Example 5, the subject matter of any one or more of Examples 1-4 optionally include wherein the ionomer comprises a perfluorinated polymer having sulfonic acid functional groups.

In Example 6, the subject matter of Example 5 optionally includes wherein the sulfonic acid functional groups form polar, hydrophilic clusters interspersed with hydrophobic semi-crystalline regions of perfluorinated polymer backbones.

In Example 7, the subject matter of Example 6 optionally includes wherein at least one of the polar, hydrophilic clusters and the hydrophobic semi-crystalline regions capture and modify lipid bilayer membranes of red blood cells causing disruption of the red blood cell membranes to effect the release of cytosolic hemoglobin proteins to the extracellular spaces.

In Example 8, the subject matter of any one or more of Examples 6-7 optionally include wherein the hydrophilic clusters and the hydrophobic semi-crystalline regions capture and modify extracellular Hb protein molecules within the fluid sample to alter the Hb-NO bond and coordination energies and stabilities of Hb protein molecules.

Example 9 is a cartridge for measuring nitric oxide (NO) concentration within a fluid sample, comprising: a cartridge body defining a flow path fluidly connected to a fluid chamber; and a sensor chip comprising at least one NO sensor positioned within the fluid chamber, each NO sensor comprising: a working electrode inducing NO molecules in the fluid sample to undergo an electrochemical oxidation reaction transferring electrons from the NO molecules to the working electrode, thereby inducing a measurable current corresponding to the NO concentration within the fluid sample, and an external membrane deposited onto the working electrode separating the fluid sample from the working electrode, wherein the external membrane comprises an ionomer selectively permitting diffusion of NO molecules through the external membrane from the fluid sample to the working electrode.

In Example 10, the subject matter of Example 9 optionally includes wherein the sensor chip further comprises: a heating element for heating fluid within the fluid chamber to a predetermined temperature between about 40° C. and about 60° C. to facilitate separation of gaseous NO molecules from their Hb-bound forms within the fluid chamber.

In Example 11, the subject matter of any one or more of Examples 9-10 optionally include wherein the ionomer comprises a perfluorinated polymer having sulfonic acid functional groups.

In Example 12, the subject matter of Example 11 optionally includes wherein the sulfonic acid functional groups form polar, hydrophilic clusters interspersed with hydrophobic semi-crystalline regions of perfluorinated polymer backbones.

In Example 13, the subject matter of any one or more of Examples 9-12 optionally include wherein the cartridge body further comprises: a sample port fluidly connected to the flow path and positioned upstream of the fluid chamber for supplying fluid to the fluid chamber; and a waste chamber fluidly connected to the flow path and positioned downstream of the fluid chamber for receiving fluid displaced from the fluid chamber.

In Example 14, the subject matter of any one or more of Examples 9-13 optionally include wherein the sensor chip further comprises: at least one interface connectable to a corresponding interface of a reader system.

Example 15 is a method of measuring nitric oxide (NO) in a sample fluid, the method comprising: loading a sample fluid into a fluid chamber having a NO sensor having a working electrode and an external membrane separating the sample fluid from the working electrode; heating the sample fluid to a predetermined temperature to facilitate liberation of NO molecules in the sample fluid proximate the external membrane, wherein the external membrane comprises an ionomer selectively permitting diffusion of NO molecules through the external membrane from the fluid sample to the working electrode to undergo an electrochemical oxidation reaction transferring electrons from the NO molecules to the working electrode to create a current; and measuring the current created by the electrochemical oxidation, wherein the current corresponds to the NO concentration within the sample fluid.

In Example 16, the subject matter of Example 15 optionally includes wherein the sample fluid is heated to a temperature between about 40° C. and about 60° C.

In Example 17, the subject matter of any one or more of Examples 15-16 optionally include wherein the ionomer comprises a perfluorinated polymer having sulfonic acid functional groups.

In Example 18, the subject matter of Example 17 optionally includes wherein the sulfonic acid functional groups comprise polar, hydrophilic clusters interspersed with hydrophobic semi-crystalline regions of perfluorinated polymer backbones.

In Example 19, the subject matter of Example 18 optionally includes wherein at least one of the polar, hydrophilic clusters or the hydrophobic semi-crystalline regions capture and modify lip bilayer membranes of red blood cells and cause disruption of the red blood cell membranes causing the release of cytosolic hemoglobin proteins to the extracellular spaces.

In Example 20, the subject matter of any one or more of Examples 18-19 optionally include wherein at least one of the polar, hydrophilic clusters or the hydrophobic semi-crystalline regions of the external membrane capture and modify extracellular Hb-protein molecules within the fluid sample to alter the Hb-NO bond, coordination energies and stabilities of Hb protein molecules.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

What is claimed is:

1. A nitric oxide (NO) sensor for measuring NO concentration within a fluid sample, the nitric oxide sensor comprising:
a working electrode inducing NO molecules in the fluid sample to undergo an electrochemical oxidation reaction transferring electrons from the NO molecules to the working electrode, thereby inducing a measurable current corresponding to the NO concentration within the fluid sample; and
an external membrane deposited on the working electrode separating the fluid sample from the working electrode, wherein the external membrane comprises an ionomer selectively permitting diffusion of NO molecules through the external membrane from the fluid sample to the working electrode.

2. The nitric oxide sensor of claim 1, further comprising:
a reference electrode having a stable reference surface interfacial potential to create a working electrode bias at the working electrode, the working electrode bias induces the NO molecules to undergo the electrochemical oxidation reaction upon diffusing through the external membrane.

3. The nitric oxide sensor of claim 2, further comprising:
an internal layer comprising a hydrophilic polymer matrix deposited over the reference electrode.

4. The nitric oxide sensor of claim 2, further comprising:
an auxiliary electrode for receiving the current generated from the NO molecules undergoing the electrochemical oxidation reaction.

5. The nitric oxide sensor of claim 1, wherein the ionomer comprises a perfluorinated polymer having sulfonic acid functional groups.

6. The nitric oxide sensor of claim 5, wherein the sulfonic acid functional groups comprise polar, hydrophilic clusters interspersed with hydrophobic semi-crystalline regions of perfluorinated polymer backbones.

7. The nitric oxide sensor of claim 6, wherein at least one of the polar, hydrophilic clusters and the hydrophobic semi-crystalline regions capture and modify lipid bilayer membranes of red blood cells causing disruption of the red blood cell membranes to effect the release of cytosolic hemoglobin proteins to the extracellular spaces.

8. The nitric oxide sensor of claim 6, wherein at least one of the polar, hydrophilic clusters and the hydrophobic semi-crystalline regions capture and modify extracellular Hb protein molecules within the fluid sample to alter the Hb-NO bond and coordination energies and stabilities of Hb protein molecules.

9. A cartridge for measuring nitric oxide (NO) concentration within a fluid sample, comprising:
a cartridge body defining a flow path fluidly connected to a fluid chamber; and
a sensor chip comprising at least one NO sensor positioned within the fluid chamber, each NO sensor comprising:
a working electrode inducing NO molecules in the fluid sample to undergo an electrochemical oxidation reaction transferring electrons from the NO molecules to the working electrode, thereby inducing a measurable current corresponding to the NO concentration within the fluid sample, and
an external membrane deposited onto the working electrode separating the fluid sample from the working electrode, wherein the external membrane comprises an ionomer selectively permitting diffusion of NO molecules through the external membrane from the fluid sample to the working electrode.

10. The cartridge of claim 9, wherein the sensor chip further comprises:
a heating element for heating fluid within the fluid chamber to a predetermined temperature between about 40° C. and about 60° C. to facilitate separation of gaseous NO molecules from their Hb-bound forms within the fluid chamber.

11. The cartridge of claim 9, wherein the ionomer comprises a perfluorinated polymer having sulfonic acid functional groups.

12. The cartridge of claim 11, wherein the sulfonic acid functional groups comprise polar, hydrophilic clusters interspersed with hydrophobic semi-crystalline regions of perfluorinated polymer backbones.

13. The cartridge of claim 9, wherein the cartridge body further comprises:
a sample port fluidly connected to the flow path and positioned upstream of the fluid chamber for supplying fluid to the fluid chamber; and
a waste chamber fluidly connected to the flow path and positioned downstream of the fluid chamber for receiving fluid displaced from the fluid chamber.

14. The cartridge of claim 9, wherein the sensor chip further comprises:
at least one interface connectable to a corresponding interface of a reader system.

15. A method of measuring nitric oxide (NO) in a sample fluid, the method comprising:
loading a sample fluid into a fluid chamber having a NO sensor having a working electrode and an external membrane separating the sample fluid from the working electrode;
heating the sample fluid to a predetermined temperature to facilitate liberation of NO molecules in the sample fluid proximate the external membrane, wherein the external membrane comprises an ionomer selectively permitting diffusion of NO molecules through the external membrane from the fluid sample to the working electrode to undergo an electrochemical oxidation reaction transferring electrons from the NO molecules to the working electrode to create a current; and
measuring the current created by the electrochemical oxidation, wherein the current corresponds to the NO concentration within the sample fluid.

16. The method of claim 15, wherein the sample fluid is heated to a temperature between about 40° C. and about 60° C.

17. The method of claim 15, wherein the ionomer comprises a perfluorinated polymer having sulfonic acid functional groups.

18. The method of claim 17, wherein the sulfonic acid functional groups comprise polar, hydrophilic clusters interspersed with hydrophobic semi-crystalline regions of perfluorinated polymer backbones.

19. The method of claim 18, wherein at least one of the polar, hydrophilic clusters or the hydrophobic semi-crystalline regions capture and modify lipid bilayer membranes of red blood cells and cause disruption of the red blood cell membranes causing the release of cytosolic hemoglobin proteins to the extracellular spaces.

20. The method of claim 18, wherein at least one of the polar, hydrophilic clusters or the hydrophobic semi-crystalline regions of the external membrane capture and modify extracellular Hb protein molecules within the fluid sample to alter the Hb-NO bond, coordination energies and stabilities of Hb protein molecules.
